SEQUENCE LISTING (28 sequences)

SEQ ID NO: 1 - protein, 16 aa, Artificial Sequence
  membrane transfer domain
AAVALLPAVLLALLAP

SEQ ID NO: 2 - protein, 12 aa, Artificial Sequence
  membrane transfer domain
AAVLLPVLLAAP

SEQ ID NO: 3 - protein, 11 aa, Artificial Sequence
  membrane transfer domain
LLHLSLLALAP

SEQ ID NO: 4 - protein, 15 aa, Artificial Sequence
  membrane transfer domain
LAVALLAAFLISAAP

SEQ ID NO: 5 - protein, 13 aa, Artificial Sequence
  membrane transfer domain
ALLAALLAAPGAA

SEQ ID NO: 6 - protein, 8 aa, Artificial Sequence
  membrane transfer domain
LIILLPVV

SEQ ID NO: 7 - protein, 16 aa, Artificial Sequence
  membrane transfer domain
AIGLLWLLPLLLSTAA

SEQ ID NO: 8 - protein, 9 aa, Artificial Sequence
  membrane transfer domain
LLLPILSSA

SEQ ID NO: 9 - protein, 11 aa, Artificial Sequence
  membrane transfer domain
LLLLLLLPLLG

SEQ ID NO: 10 - protein, 13 aa, Artificial Sequence
  membrane transfer domain
LGAALLLLPLLAA

SEQ ID NO: 11 - protein, 11 aa, Artificial Sequence
  membrane transfer domain
LLSVLLLPLLG

SEQ ID NO: 12 - protein, 11 aa, Artificial Sequence
  membrane transfer domain
LLLLPLLLLPA

SEQ ID NO: 13 - protein, 16 aa, Artificial Sequence
  membrane transfer domain
LLLAGLASLLLPGSAA

SEQ ID NO: 14 - protein, 20 aa, Artificial Sequence
  membrane transfer domain
GAAAAPLLVAVAALLLGAAG

SEQ ID NO: 15 - protein, 9 aa, Artificial Sequence
  membrane transfer domain
GGQLPLLVV

SEQ ID NO: 16 - DNA, 118 bp, Artificial Sequence
  sense strand of polynucleotide encoding NLS-kFGF4-p53 polypeptide
ttgaattcaaaaagaagagaaaggccgcggtagcgctgctcccggcggtcctgctggccttgctggcgcccgaaacattttcagacctatggaaactacttcctgaaaacaagcttaa

SEQ ID NO: 17 - DNA, 118 bp, Artificial Sequence
  antisense strand of polynucleotide encoding NLS-kFGF4-p53 polypeptide
ttaagcttgttttcaggaagtagtttccataggtctgaaaatgtttcgggcgccagcaaggccagcaggaccgccgggagcagcgctaccgcggcctttctcttctttttgaattcaa

SEQ ID NO: 18 - DNA, 38 bp, Artificial Sequence
  forward primer for synthesis of polynucleotide encoding human ubiquitin
ttggatccggtagtggaagcatgcagattttcgtgaaa

SEQ ID NO: 19 - DNA, 28 bp, Artificial Sequence
  reverse primer for synthesis of polynucleotide encoding human ubiquitin
ttgaattcaccacgaagtctcaacacaa

SEQ ID NO: 20 - DNA, 36 bp, Artificial Sequence
  forward primer for synthesis of polynucleotide encoding human p18
ttaagcttatggccgagccttgggggaacgagttgg

SEQ ID NO: 21 - DNA, 30 bp, Artificial Sequence
  reverse primer for synthesis of polynucleotide encoding human p18
ttctcgagttattgaagattttgtggctcc

SEQ ID NO: 22 - DNA, 24 bp, Artificial Sequence
  forward primer for synthesis of polynucleotide encoding human p18 site-directed mutant
cttagaggtgctaatcccgatttg

SEQ ID NO: 23 - DNA, 24 bp, Artificial Sequence
  reverse primer for synthesis of polynucleotide encoding human p18 site-directed mutant
gtctttcaaatcgggattagcacc

SEQ ID NO: 24 - DNA, 849 bp, Artificial Sequence
  polynucleotide encoding human ubiquitin-NLS-MTD-p53-p18 polypeptide
atgcagattttcgtgaaaacccttacggggaagaccatcaccctcgaggttgaaccctcggatacgatagaaaatgtaaaggccaagatccaggataaggaaggaatacctcctgatcagcagagactgatctttgctggcaagcagctggaagatggacgtactttgtctgactacaatattcaaaaggagtctactcttcatcttgtgttgagacttcgtggtggtgaattcaaaaagaagagaaaggccgcggtagcgctgctcccggcggtcctgctggccttgctggcgcccgaaacattttcagacctatggaaactacttcctgaaaacaagcttatggccgagccttgggggaacgagttggcgtccgcagctgccaggggggacctagagcaacttactagtttgttgcaaaataatgtaaacgtcaatgcacaaaatggatttggaaggactgcgctgcaggttatgaaacttggaaatcccgagattgccaggagactgctacttagaggtgctaatcccgatttgaaagaccgaactggtaatgctgtcattcatgatgcggccagagcaggtttcctggacactttacagactttgctggagtttcaagctgatgttaacatcgaggataatgaagggaacctgcccttgcacttggctgccaaagaaggccacctccgggtggtggagttcctggtgaagcacacggccagcaatgtggggcatcggaaccataagggggacaccgcctgtgatttggccaggctctatgggaggaatgaggttgttagcctgatgcaggcaaacggggctgggggagccacaaatcttcaataa

SEQ ID NO: 25 - protein, 282 aa, Artificial Sequence
  human ubiquitin-NLS-MTD-p53-p18 polypeptide
MQIFVKTLTGKTITLEVEPSDTIENVKAKIQDKEGIPPDQQRLIFAGKQLEDGRTLSDYNIQKESTLHLVLRLRGGEFKKKRKAAVALLPAVLLALLAPETFSDLWKLLPENKLMAEPWGNELASAAARGDLEQLTSLLQNNVNVNAQNGFGRTALQVMKLGNPEIARRLLLRGANPDLKDRTGNAVIHDAARAGFLDTLQTLLEFQADVNIEDNEGNLPLHLAAKEGHLRVVEFLVKHTASNVGHRNHKGDTACDLARLYGRNEVVSLMQANGAGGATNLQ

SEQ ID NO: 26 - protein, 5 aa, Artificial Sequence
  nucleus localization signal domain
KKKRK

SEQ ID NO: 27 - protein, 7 aa, Artificial Sequence
  nucleus localization signal domain
PKKKRKV

SEQ ID NO: 28 - protein, 16 aa, Artificial Sequence
  nucleous localization signal domain
KRPAATKKAGQAKKKK

(19) United States
(12) Patent Application Publication
(10) Pub. No.: US 2011/0135596 A1
(43) Pub. Date: Jun. 9, 2011

(54) FUSION PROTEIN COMPRISING UBIQUITIN OR UBIQUITIN-LIKE PROTEIN, MEMBRANE TRANSLOCATION SEQUENCE AND BIOLOGICALLY ACTIVE MOLECULE AND USE THEREOF

(75) Inventors: Jae-il LEE, Yongin-si (KR); Tae-soo LEE, Seoul (KR); Young-sun LEE, Yongin-si (KR)

(73) Assignee: SAMSUNG ELECTRONICS CO., LTD., Suwon-si (KR)

(21) Appl. No.: 12/959,965

(22) Filed: Dec. 3, 2010

(30) Foreign Application Priority Data
Dec. 4, 2009 (KR) 10-2009-0119912

(51) Int. Cl. A61K 38/20; C07K 14/00; C07K 14/62; C07K 14/52; C07H 21/00; C12N 15/63; C12N 5/10; C07K 14/525; C07K 14/54; C07K 14/555; C07K 14/56; C07K 14/565; C07K 14/57; A61K 38/21; A61K 38/22; A61K 38/19; A61K 38/25; A61K 38/27; A61K 38/28; A61K 38/29; A61K 38/24; C12P 21/02; C12N 1/21; A61P 43/00

(52) U.S. Cl. 424/85.2; 530/350; 530/399; 530/351; 536/23.4; 435/320.1; 435/325; 514/21.2; 514/5.9; 424/85.1; 424/85.4; 424/85.5; 424/85.6; 424/85.7; 514/7.6; 514/7.7; 514/9.7; 514/11.6; 514/12.8; 514/12.9; 514/11.8; 514/11.2; 514/9.6; 514/10.1; 435/69.7; 435/69.4; 435/69.5; 435/69.51; 435/69.52; 435/252.3

(57) ABSTRACT

A transmembrane fusion protein including ubiquitin or a ubiquitin-like protein, a membrane translocation sequence linked to the C-terminus of the ubiquitin or ubiquitin-like protein, and a biologically active molecule linked to the C-terminus of the membrane translocation sequence is disclosed herein. A polynucleotide encoding the transmembrane fusion protein, a recombinant expression vector including the polynucleotide sequence, a cell transformed by the recombinant expression vector, and a method of delivering the biologically active molecule into a cell using the transmembrane fusion protein are also disclosed.

### CROSS-REFERENCE TO RELATED APPLICATIONS

This application claims priority to Korean Patent Application No. 10-2009-0119912, filed on Dec. 4, 2009, and all the benefits accruing therefrom under 35 U.S.C. §119, the disclosure of which is incorporated herein in its entirety by reference.

### BACKGROUND

1. Field

The present disclosure relates to a transmembrane fusion protein including ubiquitin or ubiquitin-like protein, a membrane translocation sequence that is linked to the C-terminus of the ubiquitin or ubiquitin-like protein, and a biologically active molecule that is linked to the C-terminus of the membrane translocation sequence, a polynucleotide encoding the transmembrane fusion protein, a recombinant expression vector including the polynucleotide sequence, a cell transformed by the recombinant expression vector, and a method of delivering the biologically active molecule into a cell using the transmembrane fusion protein.

2. Description of the Related Art

Since it was reported that various diseases result from abnormal activities of a protein in cells, attempts have been made to develop drugs for treating diseases by regulating the abnormal activity of a protein. In particular, research into peptide, polypeptide and protein drugs has been carried out based on enzyme-protein or protein-protein interactions which may specifically regulate biological activity. Even though peptides, polypeptides, and proteins have superior biological selectivities and effects, it is difficult for them to penetrate a cell membrane due to their size and various biochemical properties. Since peptides, polypeptides, and proteins cannot be directly delivered into cells, their practical use as effective drugs has had limitations.

A variety of attempts have been made to deliver peptides, polypeptides, or proteins into cells. Recently, transmembrane peptides have been used to deliver peptides, polypeptides, or proteins into cells. Research into the cell-penetrating mechanisms of proteins has been carried out since it was reported in 1988 that Tat protein of HIV-1 was actively transported into a cell when used with a culture medium. Since then, diverse research has been conducted into various peptides that penetrate a cell membrane, for example, signal sequences (transmembrane sequence) or the cytoplasmic penetration sequences existing in Antp protein of a drosophila, VP22 protein of herpes simplex virus (HSV), and fibroblast growth factor (FGF).

According to recent research, a membrane translocation sequence (MTS) directly penetrates a cell membrane without using a receptor/transporter, while other cell penetrating peptides (CPPs) need to be delivered using a receptor/transporter. Thus, an MTS is not dependent on the expression of a receptor/transporter. In addition, since an MTS is transferred independently of endocytosis, an MTS is more efficiently transferred into a cell. An MTS can migrate cell-to-cell, although other CPPs cannot migrate in the same way. Due to theses features, membrane translocation sequences are attracting a lot of attention.

However, due to its hydrophobicity, it is difficult to apply an MTS in animal tests. For example, if a fusion protein including an MTS is expressed in a recombinant cell, the fusion protein including the MTS is obtained as an inclusion body in a heterogenous form which has poor stability. Further, it is difficult to obtain active fusion protein from the inclusion body.

Thus, there is still a need to develop a fusion protein including an MTS that is stably transferred into cells.

### SUMMARY

Provided are a transmembrane fusion protein including ubiquitin or a ubiquitin-like protein, a membrane translocation sequence that is linked to the C-terminus of the ubiquitin or ubiquitin-like protein, and a biologically active molecule that is linked to the C-terminus of the membrane translocation sequence, a polynucleotide encoding the transmembrane fusion protein, a recombinant expression vector including the polynucleotide sequence, a cell transformed by the recombinant expression vector, and a method of delivering the biologically active molecule into a cell using the transmembrane fusion protein.

In an embodiment, the method of delivering the biologically active molecule into a cell using the transmembrane fusion protein includes contacting the transmembrane fusion protein with a cell.

A method of producing the transmembrane fusion protein is also disclosed. In an embodiment, the method includes culturing a host cell transformed by a polynucleotide encoding the transmembrane fusion protein under conditions which allow expression of the transmembrane fusion protein; and recovering the transmembrane fusion protein from the host cell culture.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings of which:

FIG. 1 is a schematic diagram illustrating transfer of a fusion protein according to an embodiment of the present invention into a cell and/or a nucleus;

FIG. 2A to FIG. 2C show fluorescent microscopic images visualizing the in vivo distribution of a fusion protein according to an embodiment of the present invention or controls in mouse cells isolated after intraperitoneal injection of the fusion protein or controls into the mice; and

FIG. 3 is a graph showing tumor volume as a function of injection time of p53-p18 contained in a fusion protein according to an embodiment of the present invention or a control fusion protein.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

According to an embodiment of the present invention, a transmembrane fusion protein includes: ubiquitin or a ubiquitin-like protein; a membrane translocation sequence that is linked to the C-terminus of the ubiquitin or ubiquitin-like protein; and a biologically active molecule that is linked to the C-terminus of the membrane translocation sequence.

The term “transmembrane fusion protein” used herein indicates a fusion protein having the capability of delivering a biologically active molecule into a cell or a nucleus in vitro and/or in vivo. In addition, a “membrane translocation sequence (MTS)” is a polypeptide having an amino acid sequence which may penetrate a phospholipid bilayer of a cell membrane. An MTS has a single hydrophobic region at the N-terminus and is formed of relatively few amino acids (7 to 17 amino acids) in a flexible helix structure. Thus, an MTS has hydrophobicity.

The term, “link”, as used herein, shall mean the linking of two or more biomolecules so that the biological functions, activities, and/or structure associated with the biomolecules are at least retained. In reference to polypeptides, the term means that the linking of two or more polypeptides results in a fusion polypeptide that retains at least some of the respective individual activities of each polypeptide component. The two or more polypeptides may be linked directly or via a linker.

Ubiquitin (Ub) is a highly conserved protein found in all eukaryotic cells that includes 76 amino acids. Ubiquitin exhibits perfect homology among species as diversed as insects, trout, and humans. In addition, ubiquitin is stable against pH changes, not easily denatured at high temperature, and stable against proteases. Thus, if ubiquitin is fused to an MTS, the water-insolubility of the MTS may be reduced.

A ubiquitin-like protein (UbL) is a protein having properties that are similar to those of ubiquitin. Examples of a ubiquitin-like protein include Nedd8, SUMO-1, SUMO-2, NUB1, PIC1, UBL3, UBL5, and ISG15

The ubiquitin or ubiquitin-like protein in the transmembrane fusion protein may be selected from the group consisting of wild-type ubiquitin, a wild-type ubiquitin-like protein, mutant ubiquitin and a mutant ubiquitin-like protein. A mutant ubiquitin is obtained by changing the amino acid sequence of wild-type ubiquitin into another amino acid sequence. For example, a mutant ubiquitin may be prepared by replacing a Lys of wild-type ubiquitin with an Arg or by replacing the RGG residues of the C-terminus of wild-type ubiquitin with RGA residues. In mutant ubiquitins prepared by replacing Lys of wild-type ubiquitin with Arg, Lys residues that exist at the 6^{th}, 11^{th} 27^{th}, 29^{th}, 33^{rd}, 48^{th}, and 63^{rd} amino acid positions may be replaced with Arg independently or in any combination.

The ubiquitin or ubiquitin-like protein in the transmembrane fusion protein has an amino acid sequence at the C-terminus which can or cannot be cleaved by a protease. An amino acid sequence that is cleaved by a protease may be identified by searching a database that is known in the art. For example, the tool PeptideCutter, available on the internet from the ExPASy (Expert Protein Analysis System) proteomics server of the Swiss Institute of Bioinformatics may be used to identify a protease and the amino acid sequence cleaved by the protease.

In some embodiments, if the amino acid sequence recognized by a protease is contained in ubiquitin or the ubiquitin-like protein, the ubiquitin or ubiquitin-like protein in the transmembrane fusion protein may be cleaved by the protease existing in the cell after the fusion protein is transferred into the cell, so that the Ub or UbL is released from the fusion protein and the biologically active molecule may perform desirable functions. In some embodiments, if the ubiquitin or the ubiquitin-like protein contains an amino acid sequence recognized by an extracellular protease, e.g., a protease existing in the blood such a ubiquitin C-terminal hydrolase, the ubiquitin or ubiquitin-like protein in the transmembrane fusion protein may be cleaved from the transmembrane fusion protein by the extracellular protease before the fusion protein is transferred into a cell by the MTS. Even though the biologically active molecule in the transmembrane fusion protein still includes the MTS after the ubiquitin or ubiquitin-like protein is removed by proteolytic cleavage, the MTS does not affect the function of the biologically active molecule since the polypeptide sequence of the MTS is short. In some embodiments, even when the ubiquitin or ubiquitin-like protein is not cleaved from the transmembrane fusion protein by a cellular protease, the biologically active molecule may perform desirable functions.

The MTS in the transmembrane fusion protein may be any polypeptide having an amino acid sequence that may pass through a phospholipid bilayer of a cell membrane without limitation. For example, the MTS may be a polypeptide having a sequence selected from SEQ ID NOS: 1 to 15.

The “biologically active molecule” in the transmembrane fusion protein is a polypeptide or protein with a desired biological activity to target into a cell that may be conjugated to an MTS and transferred into a cell.

The biologically active molecule in the transmembrane fusion protein is a peptide or protein having a length of at least two amino acids. The polypeptide may be a protein related to cell immortality such as SV40 large T antigen and telomerase; an anti-apoptotic protein such as mutant p53 and BclxL; an antibody; a cancer gene product such as ras, myc, human papilloma virus E6/E7, and adenovirus Ela; a cell cycle regulating protein such as cyclin and cyclin-dependent kinase; a green fluorescent protein; or an enzyme such as β-galactosidase and chloramphenicol acetyl transferase, but is not limited thereto.

The biologically active molecule contained in the transmembrane fusion protein may be a hydrophilic protein, but is not limited thereto. In addition, the biologically active molecule may be a hormone, a hormone-like protein, an enzyme, an enzyme inhibitor, a signal transduction protein or parts thereof, an antibody or parts thereof, a single chain antibody, a binding protein or a binding domain thereof, an antigen, an adhesion protein, a structural protein, a regulatory protein, toxoprotein, cytokine, transcription factor, blood clotting factor, a vaccine, or the like, but is not limited thereto.

For example, the biologically active molecule contained in the transmembrane fusion protein may be p53, p18, p53-p18, p21, p25, p57, p16, p15, NIP71, neuroregulin 1, PTEN tumor suppressor, ARF tumor suppressor, APC, CD95, folliculin, MEN1, BRCA1, Von Hippel-Lindau tumor suppressor, RKIP, nm23, endostatin, insulin, insulin-like growth factor 1 (IGF-1), growth factor, erythropoietin, granulocyte-colony stimulating factor (G-CSF), granulocyte/macrophage-colony stimulating factor (GM-CSF), interferon-α, interferon-β, interferon-γ, interleukin-1α and β, interleukin-3, interleukin-4, interleukin-6, interleukin-2, epidermal growth factor (EGF), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine al, triptorelin, bivalirudin, carbetocin, cyclo-sporine, exedine, lanreotide, luteinizing hormone-releasing hormone (LHRH), nafarelin, parathyroid hormone, pramlintide, enfuvirtide (T-20), thymalfasin, or ziconotide, but is not limited thereto.

The transmembrane fusion protein may further include a nuclear localization signal domain when the biologically active molecule needs to be delivered into the nucleus.

A “nuclear localization signal domain (NLS)” is an amino acid sequence capable of passing through the nuclear membrane of a cell which is contained in proteins transported from cytoplasm to nucleus. Here, the amino acid sequence is not limited as long as the sequence possesses NLS function. The polypeptide that may be used as the NLS may be KKKRK (SEQ ID NO: 26), PKKKRKV (SEQ ID NO: 27), KRPAATKKAGQAKKKK (SEQ ID NO: 28), or the like, but is not limited thereto.

The NLS may be located in at least one region of the transmembrane fusion protein selected from the group consisting of the C-terminus of ubiquitin or the ubiquitin-like protein, the C-terminus of the MTS, and the C-terminus of the biologically active molecule.

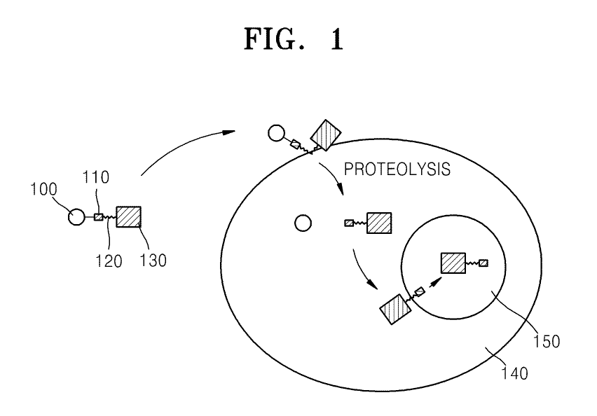

FIG. 1 schematically illustrates transfer of a fusion protein according to an embodiment of the present invention into a cell and/or a nucleus.

In a schematic diagram, if the amino acid sequence recognized by a protease is contained in ubiquitin or the ubiquitin-like protein 100, the ubiquitin or ubiquitin-like protein 100 in the transmembrane fusion protein may be cleaved by the protease existing in the cell 140 after the fusion protein is transferred into the cell 140, so that the ubiquitin or the ubiquitin-like protein 100 is released from the fusion protein and the biologically active molecule 130 may perform desirable functions in cytosol. Alternatively, the biologically active molecule 130 may perform desirable functions in nucleus 150 if the fusion protein has NLS 110. Even though the biologically active molecule 130 in the transmembrane fusion protein still includes the MTS 120 after the ubiquitin or ubiquitin-like protein 100 is removed by proteolytic cleavage, the MTS 120 does not affect the function of the biologically active molecule 130 since the polypeptide sequence of the MTS 120 is short.

“Isolated,” when used to describe the various polypeptides or polynucleotides disclosed herein, means a polypeptide or polynucleotide that has been identified and separated and/or recovered from a component of its natural environment. The term also embraces recombinant polynucleotides and polypeptides and chemically synthesized polynucleotides and polypeptides.

According to an embodiment of the present invention, there is provided a composition for delivering the biologically active molecule to a cell. The composition includes the transmembrane fusion protein disclosed herein, and a pharmaceutically acceptable carrier. The transmembrane fusion protein may be present in the composition in an amount such that the biologically active molecule is delivered in a therapeutically effective amount.

According to an embodiment of the present invention, a polynucleotide is provided which encodes a transmembrane fusion protein that includes: ubiquitin or a ubiquitin-like protein; an MTS that is linked to the C-terminus of the ubiquitin or ubiquitin-like protein; and a biologically active molecule that is linked to the C-terminus of the MTS.

A “polynucleotide” as used herein is a polymer of deoxyribonucleotides or ribonucleotides. The polynucleotide can be a single-stranded or double-stranded nucleic acid. The polynucleotide includes a RNA genome sequence, a cDNA sequence, a RNA sequence transcribed from the cDNA, and an analog of a natural polynucleotide, unless otherwise indicated herein.

The polynucleotide includes not only the nucleotide sequence encoding the amino acid sequence of the transmembrane fusion protein, but also its complementary sequence. The complementary sequence may be a perfectly complementary sequence or a substantially complementary sequence to the nucleotide sequence. That is, the complementary sequence may be a sequence that is hybridized with, for example, a nucleotide sequence that encodes an amino acid sequence of the transmembrane fusion protein under stringent conditions known in the art. Specifically, stringent conditions mean, for example, hybridization to DNA in 6×SSC at about 45° C., followed by one or more washes in 0.2×SSC/0.1% SDS at about 50° C.-65° C.

A nucleotide sequence encoding a ubiquitin or a ubiquitin-like protein contained in the transmembrane fusion protein may be derived from a mammal, for example, a human. In addition, the nucleotide sequence encoding a ubiquitin or a ubiquitin-like protein may be obtained by searching a known nucleotide sequence database, for example, the National Center for Biotechnology Information (NCBI) Entrez databases or the European Molecular Biology Laboratory-European Bioinformatics Institute (EMBL-EBI) databases.

According to another embodiment of the present invention, a recombinant vector includes: a polynucleotide encoding a transmembrane fusion protein including: ubiquitin or a ubiquitin-like protein; an MTS that is linked to the C-terminus of the ubiquitin or ubiquitin-like protein; and a biologically active molecule that is linked to the C-terminus of the MTS; and a promoter operatively linked to the polynucleotide sequence.

The term “operatively linked” used herein indicates a functional binding of a nucleotide expression controlling sequence (e.g., promoter sequence) to another nucleotide sequence, and the nucleotide expression controlling sequence may control transcription and/or translation of the other nucleotide sequence thereby.

The recombinant vector may be an expression vector that stably expresses the transmembrane fusion protein in a host cell. The expression vector may be a vector known in the art that is used to express an exogenous protein in plants, animals, or microorganisms. The recombinant vector may be formed using various methods known in the art.

The recombinant vector may be constructed for use in a prokaryotic or a eukaryotic host cell. For example, if the vector is an expression vector for use in a prokaryotic host cell, the vector may include a promoter capable of initiating transcription, such as p_{L}^{λ} promoter, trp promoter, lac promoter, tac promoter, and T7 promoter; a ribosome-binding site to initiate translation; and a transcription/translation termination sequence. When a eukaryotic cell is used as the host cell, the vector may contain an origin of replication operating a eukaryotic cell, e.g., a f1 replication origin, a SV40 replication origin, a pMB1 replication origin, an adeno replication origin, an AAV replication origin, and a BBV replication origin, but is not limited thereto. The promoter used in an expression vector for a eukaryotic host cell may be a promoter derived from a mammalian cell genome (for example, a metallothionein promoter) or a promoter derived from a mammalian virus (for examplean adenovirus anaphase promoter, vaccunia virus 7.5K promoter, a SV40 promoter, a cytomegalo virus promoter, or tk promoter of HSV). The expression vector for a eukaryotic host cell may in general also include a polyadenylated sequence as a transcription termination sequence.

According to an embodiment of the present invention, a host cell is provided which includes the polynucleotide sequence encoding the transmembrane fusion protein disclosed herein. The host cell may be a cell that is transformed by the recombinant vector including the polynucleotide encoding the transmembrane fusion protein.

The host cell may include the polynucleotide encoding the transmembrane fusion protein including: ubiquitin or ubiquitin-like protein; an MTS that is linked to the C-terminus of the ubiquitin or ubiquitin-like protein; and a biologically active molecule that is linked to the C-terminus of the MTS in a genome, or may include the recombinant vector including the polynucleotide sequence.

The host cell that can stably and continuously clone or express the recombinant vector may be any host cell that is known in the art. Examples of a prokaryotic host cell are E. coli JM109, E. coli BL21, E. coli RR1, E. coli LE392, E. coli B, E. coli X 1776, E. coli W3110, strains of Bacillus species such as Bacillus subtillis or Bacillus thuringiensis, intestinal bacteria and strains such as salmonella typhymurum, Serratia marcescens, or various Pseudomonas species. Examples of a eukaryotic host cell are Saccharomyces cerevisiae, an insect cell, a plant cell, or an animal cell such as Chinese hamster ovary (CHO), W138, BHK, COS-7, 293, HepG2, 3T3, RIN, and MDCK cell lines.

The polynucleotide or the recombinant vector including the polynucleotide may be delivered to a host cell using a delivery method known in the art. The delivery method may vary according to the host cell. If the host cell is a prokaryotic cell, a CaCl₂ method or an electroporation method may be used. If the host cell is a eukaryotic cell, microinjection, calcium phosphate precipitation, electroporation, liposome-mediated transfection, or gene bombardment may be used. However, the delivery method is not limited thereto.

The transformed host cell may be selected using a phenotype expressed by a selectable marker and a method known in the art. For example, if the selectable marker is a specific antibiotic resistance gene, the transformed host cell may be distinguished by culturing the transformed cell in a medium containing the antibiotic.

According to another embodiment of the present invention, a method of preparing a transmembrane fusion protein is disclosed. The method comprises: culturing a cell including a polynucleotide sequence encoding a transmembrane fusion protein including: ubiquitin or ubiquitin-like protein; an MTS that is linked to the C-terminus of the ubiquitin or ubiquitin-like protein; and a biologically active molecule that is linked to the C-terminus of the MTS under conditions at which the transmembrane fusion protein is expressed; and isolating transmembrane fusion protein expressed in a culture medium.

The cultivation of the transformed cell may be performed using a method known in the art. For example, the transformed cell can be inoculated into a YT liquid culture medium and cultured. When cell density reaches a predetermined level, isopropyl-beta-thio galactopyranoside (IPTG) can be added to the culture medium to induce the expression of the protein by the lacZ promoter. Subsequently, protein expressed within the cell or secreted to the culture medium may be collected.

The protein expressed within the cell or secreted to the culture medium may be isolated using a purification method that is known in the art. For example, solubility fractionation using ammonium sulfate, size classification and filtration, and various chromatography separation methods (separation according to size, charge, hydrophobicity, or hydrophilicity) may be used to obtain isolated proteins. For example, if the transmembrane fusion protein includes glutathione S-transferase (GST), the protein may be efficiently isolated using a glutathione resin column. If the transmembrane fusion protein includes 6×His sequence, a Ni²⁺-NTA His-binding resin column may be used to obtain the desired protein.

According to another embodiment of the present invention, a method of delivering a transmembrane fusion protein into a cell is provided.

The method may include contacting the transmembrane fusion protein with the cell.

Contacting the transmembrane fusion protein with the cell may be performed in vitro or in vivo. The cell contacted may be an individual cell, a cell in a tissue, a cell in an organ, or a cell in an individual. Contacting the transmembrane fusion protein with a cell can be performed directly in vitro by exposing the cell to the transmembrane fusion protein dissolved in a buffer solution. Contacting the transmembrane fusion protein with the cell in vivo may include administering the transmembrane fusion protein to a subject.

The subject may be a mammal, for example a human. Administration of the transmembrane fusion protein may be parenteral administration to an individual of the transmembrane fusion protein. The transmembrane fusion protein may be alone or in a composition with a pharmaceutically acceptable excipient. Parenteral administration may be performed using intravenous injection, subcutaneous injection, intramuscular injection, intraperitoneal injection, endodermal injection, topical administration, intranasal administration, lung administration, rectal administration, or the like. Since the administered transmembrane fusion protein includes an MTS, the biologically active molecule may be delivered into a cell contacted by the transmembrane fusion protein, which has been administered using a method described above.

The present invention will be described in further detail with reference to the following examples. These examples are for illustrative purposes only and are not intended to limit the scope of the invention.

### Example 1

### Preparation of Expression Vector for a Transmembrane Fusion Protein Including Ubiquitin, NLS, MTS, and p53-p18

An expression vector was prepared for producing a transmembrane fusion protein including the hydrophilic polypeptide ubiquitin, a NLS, an MTS, and the biologically active polypeptide p53-p18 which are sequentially linked

In order to produce the transmembrane fusion protein, a pET-NLS-kFGF4-p53 expression vector was prepared that included a polynucleotide sequence encoding the NLS polypeptide sequence KKKRK (SEQ ID NO: 26), a polynucleotide sequence encoding a known MTS sequence, the leader sequence of kFGF4 growth factor (AAVALLPAVLLALLAP; SEQ ID NO: 1), and a polynucleotide sequence encoding the N-terminal fragment of p53 polypeptide (mdm2 binding domain). The expression vector was prepared by annealing the sense and antisense strands of oligonucleotide encoding NLS-kFGF4-p53 polypeptide (sense strand: 5′gttgaattcaaaaagaagagaaaggccgcggtagcgctgctcccggcggtcctgctggccttgctggcgcccgaaacat tttcagacctatggaaactacttcctgaaaacaagcttaa3′; SEQ ID NO: 16, antisense strand: 5′ ttaagcttgttttcaggaagtagtttccataggtctgaaaatgtttcgggcgccagcaaggccagcaggaccgccgggagc agcgctaccgcggcctttctcttctttttgaattcaa3′; SEQ ID NO: 17), and then cleaving the annealed product with the restriction enzymes EcoRI and Hind III. The cleaved fragments were inserted into the vector pET22b that was cleaved using the same restriction enzymes.

Then, polymerase chain reaction (PCR) was used to amplify synthetic polynucleotides of the known cDNA sequence of human ubiquitin and the known cDNA sequence of human p18 (CDKN2C). In the PCR amplification of the human ubiquitin polynucleotide fragment, gttggatccggtagtggaagcatgcagattttcgtgaaaa (SEQ ID NO: 18) and 5′ ttgaattcaccacgaagtctcaacacaa3′ (SEQ ID NO: 19) were used as the forward and the reverse primers, respectively. The forward and reverse primers each included restriction sites for BamHI and EcoRI. In the PCR amplification of the human p18 polynucleotide fragment, 5′ ttaagcttatggccgagccttgggggaacgagttgg3′ (SEQ ID NO: 20) was used as the forward primer, and 5′ ttctcgagttattgaagattttgtggctcc3′ (SEQ ID NO: 21) was used as the reverse primer. The forward and reverse primers each included restriction sites for HindIII and XhoI. Then, to improve the hydrophilicity of the p18, the codon for the 71^{st} amino acid, phenylalanine (F), was replaced with the codon for asparagine (N) using 5′ cttagaggtgctaatcccgatttg3′ (SEQ ID NO: 22) as the forward primer and 5′ gtctttcaaatcgggattagcacc3′(SEQ ID NO: 23) as the reverse primer to perform site-directed mutagenesis of the nucleic acid sequence. The mutant was named “p18(F71N)”.

PCR was performed by a known method in the art using a GeneAmp PCR System 9700 (Applied Biosystem). The PCR products were purified using a QIAquick Multiwell PCR Purification kit (Qiagen) and each was cloned into a pGEM-T Easy vector (Promega). Then, for each recombinant pGEM-T Easy vector created, a competent cell (E. Coli DH5α) was transformed with the vector, and the plasmid was subsequently isolated from a culture of the transformed cell using QIAprep Spin Miniprep kit (Qiagen).

The pGEM-T Easy vector including the cDNA of human ubiquitin was cleaved using BamHI and EcoRI, and the pGEM-T Easy vector including the cDNA of human p18(F71N) was cleaved using Hind III and XhoI. Then, both fragments were inserted into the pET-NLS-kFGF4-p53 expression vector. This final vector prepared, capable of expressing the human ubiquitin-NLS-MTS-p53-p18(F71N) fusion protein, was named pET-Ub-NLS-kFGF4-p53-p18(F71N). The polypeptide sequence of the human ubiquitin-NLS-MTS-p53-p18 fusion protein inserted into the pET vector and polynucleotide sequence encoding the polypeptide sequence are shown as SEQ ID NOS: 25 and 24, respectively.

In the polynucleotide sequence of SEQ ID NO: 24, the 1^{st} to 228^{th} nucleotides encode human ubiquitin, the 235^{th} to 249^{th} nucleotides encode the NLS, the 250^{th} to 297^{th} nucleotides encode the KFG4 leader, the 298^{th} to 336^{th} nucleotides encode p53, and the 343^{rd} to 849^{th} nucleotides encode p18. The locations of each polypeptide in the amino acid sequence, as shown in SEQ ID NO: 25, may be inferred from the corresponding locations in the nucleotide sequence of SEQ ID NO: 24.

A control transmembrane fusion protein expression vector, analogous to pET-Ub-NLS-kFGF4-p53-p18(F71N) vector, but lacking ubiquitin at the NH2-terminus of the fusion protein, was also prepared as described above. The control fusion protein vector is denoted as pET-NLS-kFGF4-p53-p18(F71N) vector and the control fusion protein is denoted as NLS-kFGF4-p53-p18(F71N).

For convenient purification of these fusion proteins by affinity purification, a 6×His tag was engineered into the fusion proteins at the N-terminus of the ubiquitin, with a TEV enzyme cleavage site between the 6×His tag and the ubiquitin. The incorporation of this 6×His tag was solely for ease of purification. Other such affinity tags known in the art could be used as alternatives to incorporate into the fusion protein to permit affinity purification.

### Example 2

### Expression and Purification of the Transmembrane Fusion Protein

The transmembrane fusion protein was over-expressed in E. coli BL21(DE3) transformed with the pET-Ub-NLS-kFGF4-p53-p18(F71N) vector prepared according to Example 1. The transformed bacteria were grown in YT culture medium. When the optical density was 0.5 at an absorbance of 600 nm, 0.5 mM IPTG was added to the culture medium, and the transformed E. coli BL21(DE3) was further cultured at 18° C. for 16 hours. The cultured cells were sonicated in a 50 mM Tris-HCl buffer solution (pH 8.0) including 5% glycerol, 5 mM β-mercaptoethanol, 0.2% Triton X-100 and 0.2 M NaCl, and then centrifuged to obtain the supernatant (10,000 g). The supernatant was applied to a Ni²⁺-NTA superflow column (Qiagen) in equilibrium with the buffer solution, washed using a washing buffer solution (50 mM Tris-HCl, pH 8.0, 5% glycerol, 5 mM β-mercaptoethanol, 0.2% Triton X-100 and 1 M NaCl) having a volume 5 times that of the column, and eluted using an elution buffer solution (50 mM Tris-HCl, pH 8.0, 5% glycerol, 5 mM β-mercaptoethanol, 0.2% Triton X-100 and 0.2 M NaCl). The fractions including the fusion proteins were collected, and salts contained in the fractions were removed using Amicon Ultra-15 Centrifugal Filter (Milipore), and then the fractions were concentrated. The concentration of the purified protein was quantified using bovine serum albumin (BSA) as a standard.

Expression and purification properties of the fusion protein including ubiquitin and the fusion protein not including ubiquitin (control) were determined The results are shown in Table 1 below. The fusion protein including ubiquitin according to the present embodiment showed a high expression amount, and excellent hydrophilicity, yield, concentration, and purity.

TABLE 1

| | Fusion protein | Fusion protein |
|---|---|---|
| | not including | including |
| | ubiquitin (control) | ubiquitin |
| Expression amount | 50 mg/l | 300 mg/l |
| Expression status | Inclusion body | soluble |
| Purification yield | 20 mg/l | 200 mg/l |
| Concentration | 1.2 mg/ml | 14 mg/ml |
| Purity | 80% | 95% |

### Example 3

### Characterization of the Cell-Penetrating Property and Stability of the Transmembrane Fusion Protein

The cell-penetrating property of the transmembrane fusion protein prepared in Example 2 was measured in an animal model.

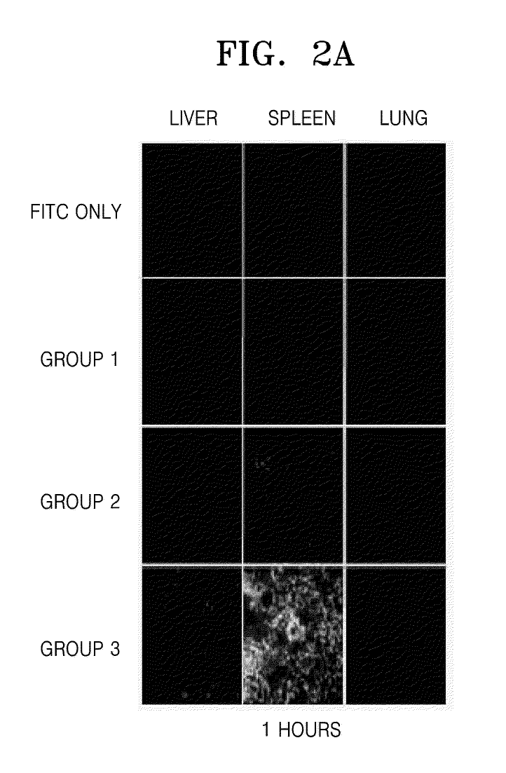

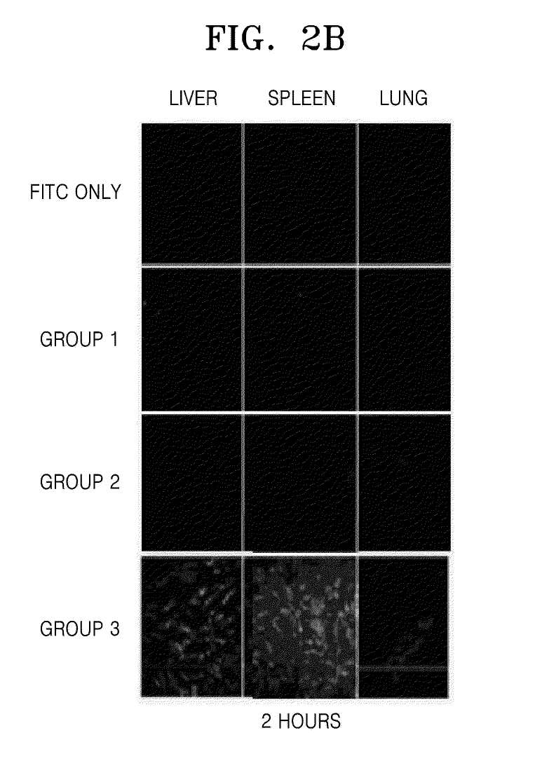

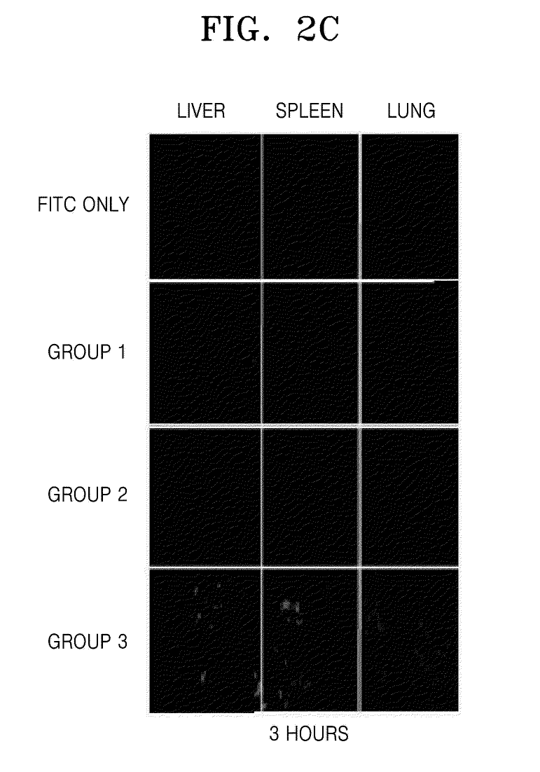

Seven week old nude mice (Balb/c mouse, Orient Bio, Inc., Korea) that were immunodeficient due to mutation in the major histocompatibility complex (MHC) were used as the animal model. Three groups of 3 mice were intraperitoneally injected with six hundred micrograms of a fusion protein at 1 μg/μl. The fusion proteins used for injection to a group of mice were the fusion protein including ubiquitin (Ub-NLS-kFGF4-p53-p18(F71N) (Group 3), the fusion protein not including ubiquitin (NLS-kFGF4-p53-p18(F71N) (Group 2), and a fusion protein including only p53-p18 (p53-p18(F71N), (Group 1). As an additional control, a fourth group of 3 mice were intraperitoneally injected with 600 μl of a Tris buffer solution including FITC (FITC). Then, cells were obtained from the liver, spleen, and lung of one of the 3 mice at 1, 2 and 3 hours, respectively, after the injection. After washing the cells twice, the cells were stained at 4° C. for 1 hour with a mouse monoclonal anti-p18 antibody (Thermo Scientific Pierce, U.S.A.) diluted to a 1:100 ratio (v/v) with a phosphate buffer solution (PBS) including 0.1% bovine serum albumin and 0.05% sodium azide. Then, the cells were washed twice, and incubated with an FITC-binding secondary goat anti-mouse IgG-HRP (Santa Cruz Biotechnology, U.S.A.) diluted in PBS including 0.1% bovine serum albumin at 4° C. for 30 minutes. The cells were washed twice, and the in vivo distribution was visualized using a fluorescence microscope (Nikon, Japan). Representative images are shown in FIG. 2A to FIG. 2C.

As shown in FIG. 2A to FIG. 2C, the amount of the fusion protein including ubiquitin present in spleen cells was larger than the amount of the fusion protein not including ubiquitin under the same condition. This indicates that the fusion protein including ubiquitin was transferred into the cell more efficiently. Thus it was determined that the transmembrane fusion protein including ubiquitin, after transfer into the cell, stably exists within the cell for a long period of time.

### Example 4

### Determination of the Therapeutic Effect of the Transmembrane Fusion Protein in a Cancer Cell Animal Model

The therapeutic effect of p53-p18 protein administered in vivo in the form of the transmembrane fusion protein prepared as in Example 2 was determined using a xenograft mouse model transplanted with a colon cancer cell line (Wild-type p53-positive HCT116), Korean Cell Line Bank (KCLB).

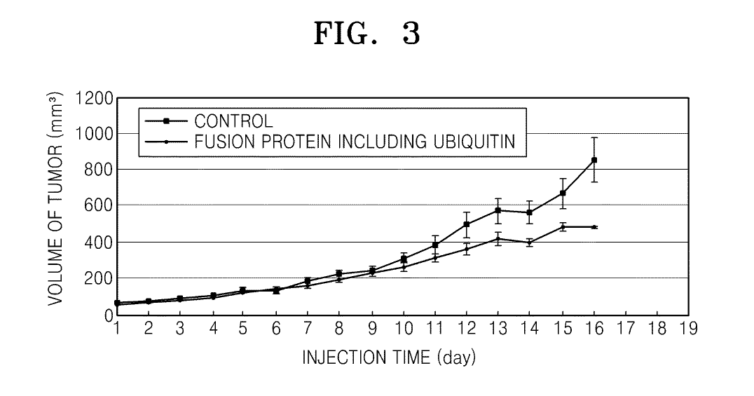

Fifty microliters of colon cancer cells HCT116 (1×10⁷) were subcutaneously injected into 6 week old female BALB/c nude mice. After 4 to 5 weeks, tumors were found in the mice. When the size of a tumor (width²×length/2) measured using a vernier caliper was 80 mm³, 200 μg of the control fusion protein (not including ubiquitin) or the fusion protein including ubiquitin were intravenously injected into 10 mice at 1 μg/μl daily for 16 days. Tumor size was monitored daily over the 16 day period. FIG. 3 shows a graph of tumor volume as a function of time after injections were begun.

Referring to FIG. 3, the injected fusion protein including ubiquitin inhibited tumor growth relative to the size of tumors observed in animals injected with the control fusion protein. By day 16, average tumor size in the group injected with the ubiquitin containing fusion protein was about 46% smaller than that observed in the group injected with the control fusion protein.

### Example 5

### Expression and Purification of Additional Transmembrane Fusion Proteins with p53-p18 as the Biologically Active Molecule

Other transmembrane fusion protein expression vectors are prepared in a similar manner as described above in Example 1 by changing the order of the modules in the sequence of the transmembrane fusion protein, for example, locating the NLS between kFGF4 and p53, replacing the RGG at the C-terminus of the ubiquitin with RGA, or replacing each of the seven Lys in ubiquitin with Arg.

Additional transmembrane fusion protein expression vectors analogous to pET-Ub-NLS-kFGF4-p53-p18(F71N) for a fusion protein with p53-p18 as the biologically active molecule are prepared replacing the kFGF4 MTS in the encoded fusion protein with another MTS. A transmembrane fusion protein expression vector for a fusion protein with p53-p18 as the biologically active molecule is made encoding an MTS selected from SEQ ID NOS: 2-15.

Each of these transmembrane fusion proteins is expressed and isolated as described in Example 2.

### Example 6

### Expression and Purification of Transmembrane Fusion Proteins Including Alternate Biologically Active Molecules

Additional transmembrane fusion protein expression vectors are made as described in Example 1, each including the coding sequence of an alternate biologically active molecule instead of p53-p18. For each of these recombinant vectors prepared, a polynucleotide fragment of the known cDNA sequence of the biolgically active molecule is synthesized by PCR for subsequent cloning into the expression vector as described above and is cloned into the expression vector using methods known in the art.

A transmembrane fusion protein expression vector is made for each of the following biologically active molecules: neuroregulin 1, PTEN tumor suppressor, ARF tumor suppressor, APC, CD95, folliculin, MEN1, BRCA1, Von Hippel-Lindau tumor suppressor, RKIP, nm23, endostatin, insulin, insulin-like growth factor 1 (IGF-1), growth factor, erythropoietin, granulocyte-colony stimulating factor (G-CSF), granulocyte/macrophage-colony stimulating factor (GM-CSF), interferon-α, interferon-β, interferon-γ, interleukin-1α and β, interleukin-3, interleukin-4, interleukin-6, interleukin-2, epidermal growth factor (EGF), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclo-sporine, exedine, lanreotide, luteinizing hormone-releasing hormone (LHRH), nafarelin, parathyroid hormone, pramlintide, enfuvirtide (T-20), thymalfasin, and ziconotide.

Each of these transmembrane fusion proteins is expressed and isolated as described in Example 2.

The terminology used herein is for the purpose of describing particular embodiments only and is not intended to be limiting of the invention. The terms “a” and “an” do not denote a limitation of quantity, but rather denote the presence of at least one of the referenced item. The terms “comprising”, “having”, “including”, and “containing” are to be construed as open-ended terms (i.e. meaning “including, but not limited to”). The modifier “about” used in connection with a quantity is inclusive of the stated value and has the meaning dictated by the context (e.g., includes the degree of error associated with measurement of the particular quantity),

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. No language in the specification should be construed as indicating any non-claimed element as essential to the practice of the invention as used herein.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. An isolated transmembrane fusion protein comprising:
ubiquitin or a ubiquitin-like protein;
a membrane translocation sequence linked to the C-terminus of the ubiquitin or ubiquitin-like protein; and
a biologically active molecule linked to the C-terminus of the membrane translocation sequence.

2. The transmembrane fusion protein of claim 1, wherein the ubiquitin or ubiquitin-like protein is a mutant.

3. The transmembrane fusion protein of claim 2, wherein the mutant ubiquitin has Arg replacing Lys in an amino acid sequence of wild-type ubiquitin.

4. The transmembrane fusion protein of claim 1, wherein the ubiquitin or ubiquitin-like protein has a protease recognition amino acid sequence that is cleaved by a protease.

5. The transmembrane fusion protein of claim 1, wherein the ubiquitin or ubiquitin-like protein is a ubiquitin-like protein selected from the group consisting of Nedd8, SUMO-1, SUMO-2, NUB1, PIC1, UBL3, UBL5, and ISG15.

6. The transmembrane fusion protein of claim 1, wherein the membrane translocation sequence comprises a polypeptide of a sequence selected from the group consisting of SEQ ID NOS: 1 to 15.

7. The transmembrane fusion protein of claim 1, wherein the biologically active molecule is selected from the group consisting of p53, p18 (CDKN2C), neuroregulin 1, PTEN tumor suppressor, ARF tumor suppressor, APC, CD95, folliculin, MEN1, BRCA1, Von Hippel-Lindau tumor suppressor, RKIP, nm23, endostatin, insulin, insulin-like growth factor 1 (IGF-1), growth factor, erythropoietin, granulocyte-colony stimulating factor (G-CSF), granulocyte/macrophage-colony stimulating factor (GM-CSF), interferon-α, interferon-β, interferon-γ, interleukin-1α and β, interleukin-3, interleukin-4, interleukin-6, interleukin-2, epidermal growth factor (EGF), calcitonin, adrenocorticotropic hormone (ACTH), tumor necrosis factor (TNF), atobisban, buserelin, cetrorelix, deslorelin, desmopressin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclo-sporine, exedine, lanreotide, luteinizing hormone-releasing hormone (LHRH), nafarelin, parathyroid hormone, pramlintide, enfuvirtide (T-20), thymalfasin, and ziconotide.

8. The transmembrane fusion protein of claim 1, further comprising a nuclear localization signal domain.

9. The transmembrane fusion protein of claim 8, wherein the nuclear localization signal domain comprises a polypeptide having SEQ ID NO: 26.

10. An isolated polynucleotide encoding the transmembrane fusion protein of claim 1.

11. The polynucleotide of claim 10, wherein the encoded membrane translocation sequence comprises a polypeptide with a sequence selected from the group consisting of SEQ ID NOS: 1 to 15.

12. The polynucleotide of claim 10, wherein the encoded transmembrane fusion protein further comprises a nuclear localization signal domain.

13. The polynucleotide of claim 12, wherein the nuclear localization signal domain comprises a polypeptide having SEQ ID NO: 26.

14. A recombinant vector comprising: the polynucleotide of claim 11; and a promoter operatively linked to the polynucleotide.

15. The recombinant vector of claim 14, wherein the encoded membrane translocation sequence comprises a polypeptide with a sequence selected from the group consisting of SEQ ID NOS: 1 to 15.

16. The recombinant vector of claim 14, wherein the encoded transmembrane fusion protein further comprises a nuclear localization signal domain.

17. The recombinant vector of claim 16, wherein the nuclear localization signal domain comprises a polypeptide having SEQ ID NO: 26.

18. A host cell transformed by the polynucleotide of claim 10.

19. A method of delivering a transmembrane fusion protein to a cell, the method comprising
contacting the transmembrane fusion protein of claim 1 with a cell.

20. The method of claim 19, wherein contacting the cell is performed in vitro or in vivo.

21. The method of claim 20, wherein contacting the transmembrane fusion protein with the cell is performed in vivo and comprises
administering the transmembrane fusion protein to a subject.

22. A method of producing a transmembrane fusion protein, comprising.
culturing the host cell of claim 18 under conditions which allow expression of the transmembrane fusion protein; and
recovering the transmembrane fusion protein from the host cell culture.

23. The method of claim 22, wherein the host cell is a bacterial host cell.
